(19) United States
(12) Patent Application Publication
(10) Pub. No.: US 2018/0221103 A1
(43) Pub. Date: Aug. 9, 2018

(54) Medical Instrument Hygienic System

(71) Applicant: Mohamed Ghiath Bayasi, Bloomfield Hills, MI (US)

(72) Inventor: Mohamed Ghiath Bayasi, Bloomfield Hills, MI (US)

(73) Assignee: B&B Medical Products, LLC, Bloomfield Hills, MI (US)

(21) Appl. No.: 15/882,628

(22) Filed: Jan. 29, 2018

Related U.S. Application Data
(60) Provisional application No. 62/456,083, filed on Feb. 7, 2017.
(60) Provisional application No. 62/459,156, filed on Feb. 15, 2017.

(51) Int. Cl. A61B 50/22; B65D 83/08

(52) U.S. Cl. CPC A61B 50/22; A61B 2050/005; B65D 83/0894

(57) ABSTRACT

A system for maintaining cleanliness of instruments contains a housing containing an exterior and an interior, the exterior optionally configured to mount to a surface. A rack contains at least one hangar and is contained within the housing. A plurality of flexible plastic covers is also contained within the housing wherein each cover is configured to hang on the rack.

### CROSS-REFERENCE TO RELATED APPLICATIONS

The present application claims the benefit of U.S. Provisional Application No. 62/456,083 having a filing date of Feb. 7, 2017, and of U.S. Provisional Application No. 62/459,156 having a filing date of Feb. 15, 2017, the teachings of each application herein incorporated by reference.

### BACKGROUND OF THE INVENTION

In various industries, it is desirable to provide a cover to maintain the cleanliness of different instruments during the use thereof. For example, in the medical industry, it is necessary to keep the equipment clean from one use to the next, and to do this without the need to extensively clean the equipment after each use. Accordingly, it would be an improvement in the art to provide a solution to the need to clean a medical instrument after each use. Furthermore, it would be an improvement in the art to provide a system to maintain the cleanliness of an instrument, such as a stethoscope, and readily provide the system to users of the stethoscope, or other instrument, for example.

### SUMMARY OF THE INVENTION

The above-referenced concerns are resolved by a system for maintaining cleanliness of various medical or other instruments that contains a housing containing an exterior and an interior, the exterior optionally and preferably configured to mount to a surface such as with two-sided adhesive tape, for example; a rack containing at least one hangar such as a dowel or pin, the rack contained within the housing; and a plurality of flexible plastic or polymeric covers, wherein each cover is preferably configured to hang on the at least one hangar. The invention may further be distinguished wherein each cover of the plurality of flexible plastic covers contains a first sleeve containing a first closed end and a first open end for receipt of an instrument, and, a second sleeve containing a second closed end attached to and adjacent to the first open end, and, a second open end opposite to the second closed end (that is,at the opposite end of the second sleeve) for opening the first open end.

### BRIEF DESCRIPTION OF THE DRAWINGS

FIG. 1 illustrates an exterior of a housing of a hygienic system in accordance with the present invention.

FIG. 2 illustrates a packing material used to retain the orientation of polymeric sleeves within a housing, in accordance with the present invention.

FIG. 3 illustrates the rear of the housing of FIG. 2, containing adhesive strips for affixing to a desired structure.

FIG. 4 illustrates an exemplary sleeve containing a first polymeric sleeve and a second polymeric sleeve.

FIG. 5 illustrates a stethoscope inserted within a polymeric sleeve, in accordance with the present invention.

FIG. 6 illustrates a suspended polymeric sleeve as shown in FIG. 4, containing an inner sleeve for insertion of a stethoscope and an outer sleeve for insertion of a user's hand, in accordance with the present invention.

FIG. 7 illustrates a preferred pair of dowels wherein each dowel contains an upwardly angled end for retention of polymeric covers hung on the pair of dowels.

FIG. 8 illustrates a perspective view of a retainer or bracket for containment of a housing containing a plurality of polymeric sleeves.

FIG. 9 illustrates a front view of a retainer or bracket for containment of a container or housing of polymeric sleeves.

FIG. 10 illustrates a frontal perspective view of a container of polymeric sleeves housed within a suspended retainer or bracket, in accordance with the present invention.

FIG. 11 illustrates a rear perspective view of a container of polymeric sleeves housed within a suspended retainer or bracket, in accordance with the present invention.

### DETAILED DESCRIPTION OF THE PREFERRED EMBODIMENTS

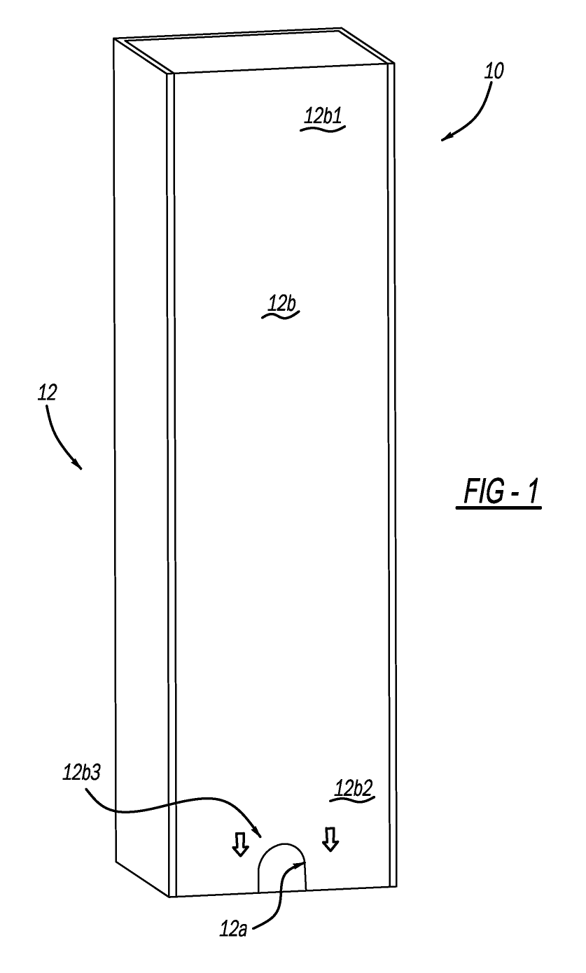

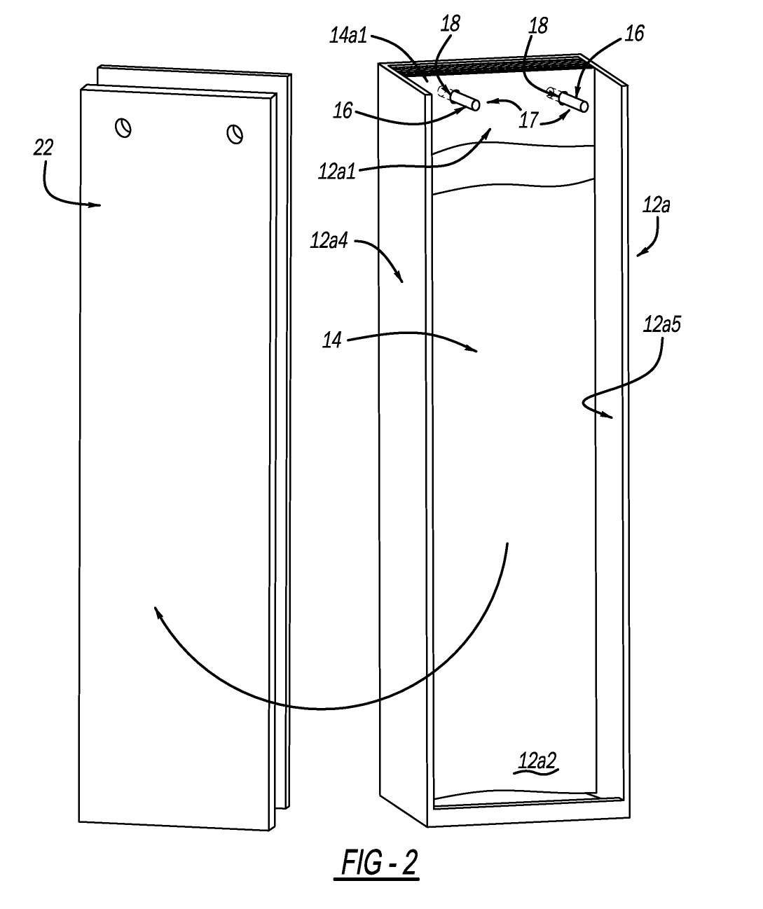

As shown in FIGS. 1-4, a medical instrument hygienic system 10, made in accordance with the present invention, includes a housing 12, a plurality of flexible plastic or polymeric covers 14 stacked and hung or retained within the housing 12, and an optional packing material 22 to ensure that the polymeric covers 14 retain their suspension within the housing 12 while being shipped. The housing 12 contains an inner compartment 12a and an outer compartment 12b, wherein the outer compartment 12b encases and preferably is slidably received over the length of the inner compartment 12a. The inner compartment 12a contains a top 12a1 and a bottom 12a2. The outer compartment 12b contains a closed top 12b1 adjacent to the top 12a1 of the inner compartment 12a, and, an open bottom 12b2 adjacent to the bottom 12a2 of the inner compartment 12a. A thumb portion 12b3 is positioned at the bottom 12b2 of the outer compartment 12b, and as shown in FIGS. 1 and 2, is used to grip the outer compartment 12b so that the outer compartment 12b may be released from the inner compartment 12a. The inner compartment 12a contains a peripheral wall 12a4 preferably shaped as a trapezoid, for example, but it may also be shaped as otherwise desired, as a rectangle for example. The peripheral wall 12a4 is preferably shaped to permit slidable engagement of the outer compartment 12b over the peripheral wall 12a4.

The inner compartment 12a essentially defines an inner trough 12a5 for storage of the polymeric covers 14. At least one dowel or hanger, and preferably a pair of dowels, hangars, or pins 16 are fixed at the top end 12a1 of the inner compartment 12a, within the inner trough 12a5, for hanging the plurality of covers 14 thereon. The hangar or hangars 16 taken together constitute a rack 17 for the plurality of covers 14. When stored within the inner trough 12a5, each polymeric cover 14 contains a top portion 14a1 adjacent the top 12a1 of the inner compartment 12a. A plurality of apertures 18, for example four as shown in the figures, are formed within the open top portion 14a1 of each cover 14 within the plurality of flexible polymeric covers 14. As shown in the Figures, the plurality of polymeric covers 14 are suspended or hung within the trough 12a5 by inserting the dowel(s) 16 through corresponding apertures 18 formed in the covers 14. It can be seen that a left dowel 16 is inserted through left-most apertures 18 on each cover 14 and a right dowel 16 through right-most apertures 18 on each cover 14. As a result, the plurality of covers 14 are then suspended when the inner compartment 12a is opened and vertically displayed on a wall for example.

As shown in FIG. 7, each dowel 16 may contain an outer angled end 16a/16b, wherein the upward angle of each angled end 16a or 16b results in a retentive advantage wherein the polymeric covers 14 may be suspended on the dowels 16 without inadvertently slipping off the outer end(s).

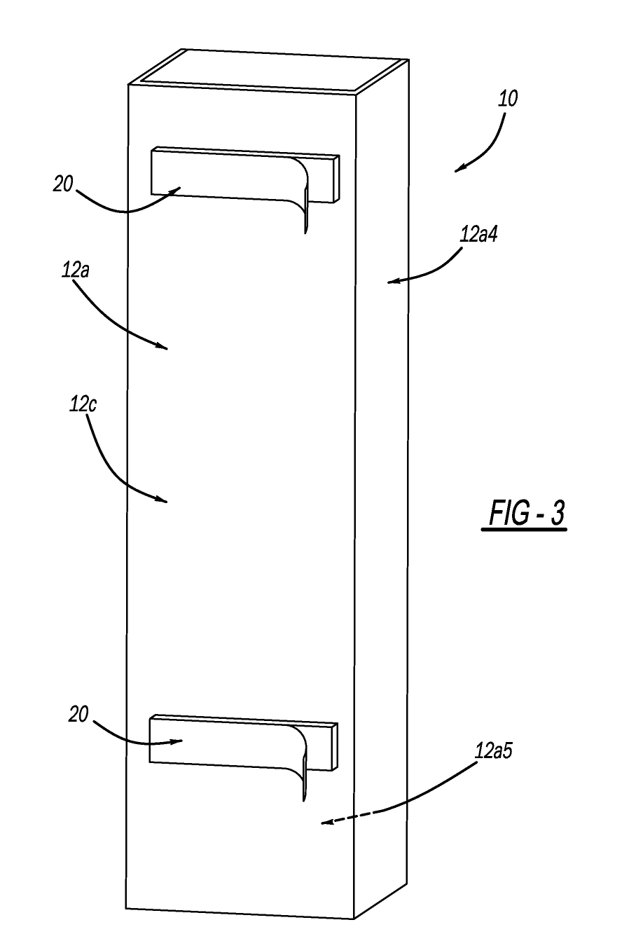

As shown in FIG. 3, a rear portion 12c of the inner compartment 12a may contain one or more adhesive backing strips 20, for adhering the inner compartment 12a to a desired structure such as a wall of a medical office or a cabinet surface, for example.

In operation, the hygienic system 10 may be opened or accessed by grabbing the thumb portions 12b3 and gripping the inner compartment 12a as the outer compartment 12b is slidably removed from over the inner compartment 12a. The thumb portion 12b3 permits opening of the housing 12 without disturbing the cleanliness and order of the inner contents. As a consequence, the inner compartment 12a is opened to reveal the stored plurality of polymeric covers 14, pre-hung on the dowel(s) or hanger(s) 16 as explained above. The inner compartment 12a may then be vertically hung on the wall by orienting the top 12a1 of the inner compartment 12a and the bottom 12a2 appropriately, and then by removing the backing on the adhesive strips 20 and affixing the inner compartment 12a onto a wall or other rigid structure, such as a cabinet door for example. Once affixed to a standing structure such as the wall or cabinet door, the open inner compartment 12a with the pre-hung polymeric covers 14 provides ready access to the each of the polymeric sleeves. As a physician or medical attendant requires a sleeve to maintain a hygienic field, for example, a polymeric cover 14 may be quickly removed from the dowels 16 by simply pulling a cover 14 off of the pair of dowels 16. The cover 14 may then be slidably applied over a medical instrument such as a stethoscope for example, to ensure the hygienic integrity of the instrument as it is applied to a patient.

The materials used for the system 10 are readily available. For example, the housing 12 may be made of cardboard, plastic, or other rigid materials. The dowels 16 may be made from wood, polymers, or other rigid materials. The flexible polymeric covers 14 may be made in a known manner, while integrating the novel features of the present invention, including the dowel apertures 18 for example. It will be appreciated that the flexible polymeric covers 14 may be made from flexible materials such as plastic sheeting, thin enough to ensure the efficacy of the instrument that is protected by the cover 14. Other suitable materials are also contemplated.

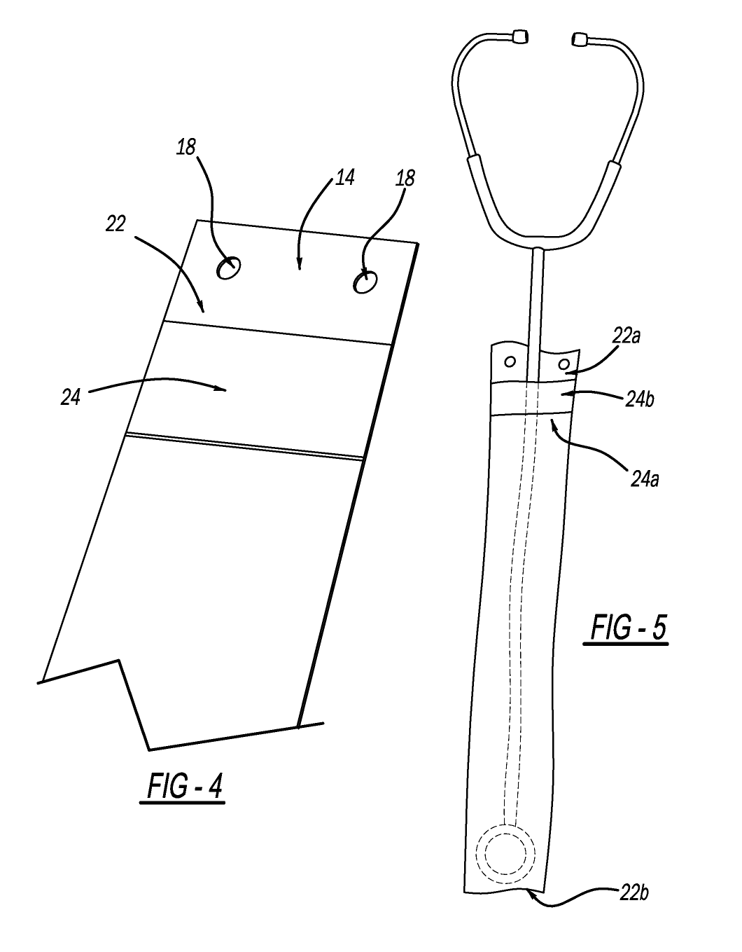

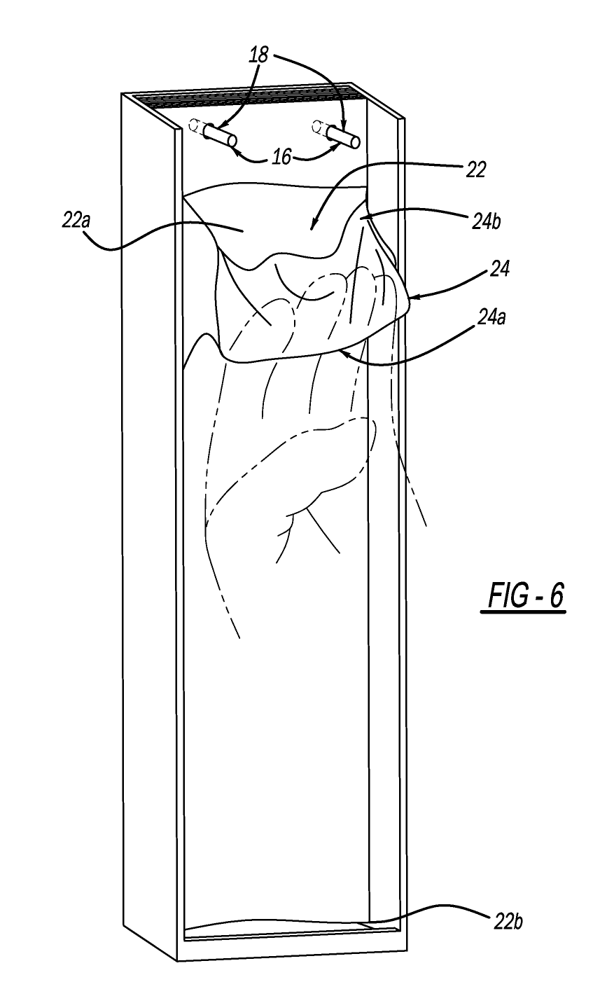

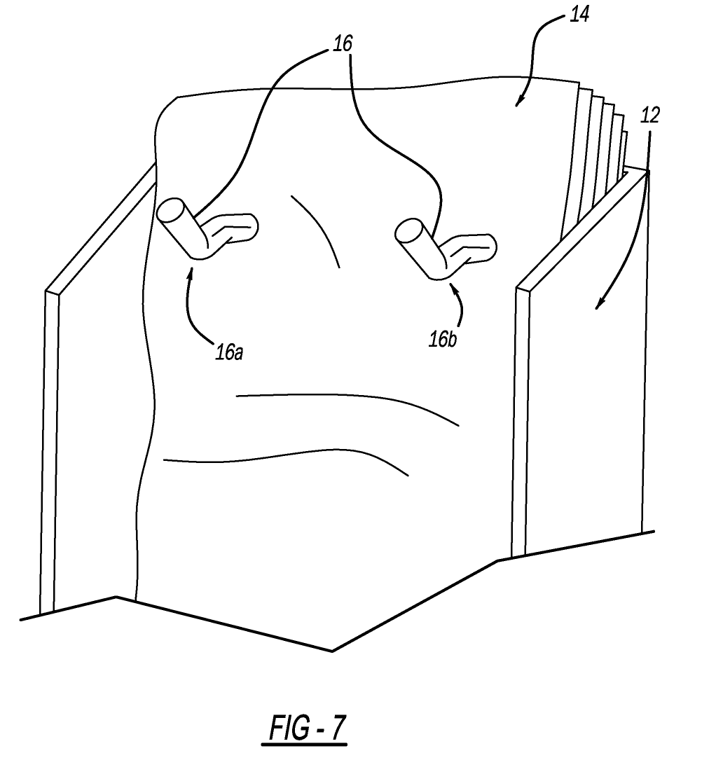

In yet another aspect of the invention, and as shown in FIG. 4, the polymeric covers 14 are each formed with a first sleeve 22 for handling of the medical instrument or other article, and, a second sleeve 24 that facilitates opening of the first sleeve 22. The first sleeve 22 is formed with a first open end 22a and a first closed end 22b for respectively accessing and closing the interior of the first sleeve 22. The second sleeve 24 is formed at or adjacent to the opening 22a of the first sleeve 22, and constitutes a fold or second closed end 24b that is formed adjacent to the opening 22a of the first sleeve 22. A second open end 24a is formed opposite the second closed end 24b. Accordingly, the second sleeve 24 is formed such that a user's hand or finger may be inserted within the second open end 24a of the sleeve 24 to thereby enable opening of the first sleeve 22 without ever touching the surface of the first sleeve 22. Once the first sleeve 22 is opened, an article or medical instrument such as a stethoscope may be inserted within the first sleeve 22 without contamination from the user's hands. As will be evident from the Figures, the second sleeve 24 may be pulled by the user's hand to open up the first sleeve 22, and then the medical instrument may be inserted into the first sleeve 22, prior to removing the polymeric cover 14 from the dowels 16. The ability to lower the stethoscope or instrument into the first sleeve as it is suspended upon the dowels 16, ensures minimal if any contact of the stethoscope and an outer portion of the first sleeve. In this way, the cleanliness of the outer portion of the first sleeve, as it contacts a patient, is assured. If desired, of course, the polymeric cover 14 may first be removed from the hangar(s) or dowel(s) 16, and then the instrument such as a stethoscope may be lowered within the open sleeve 22.

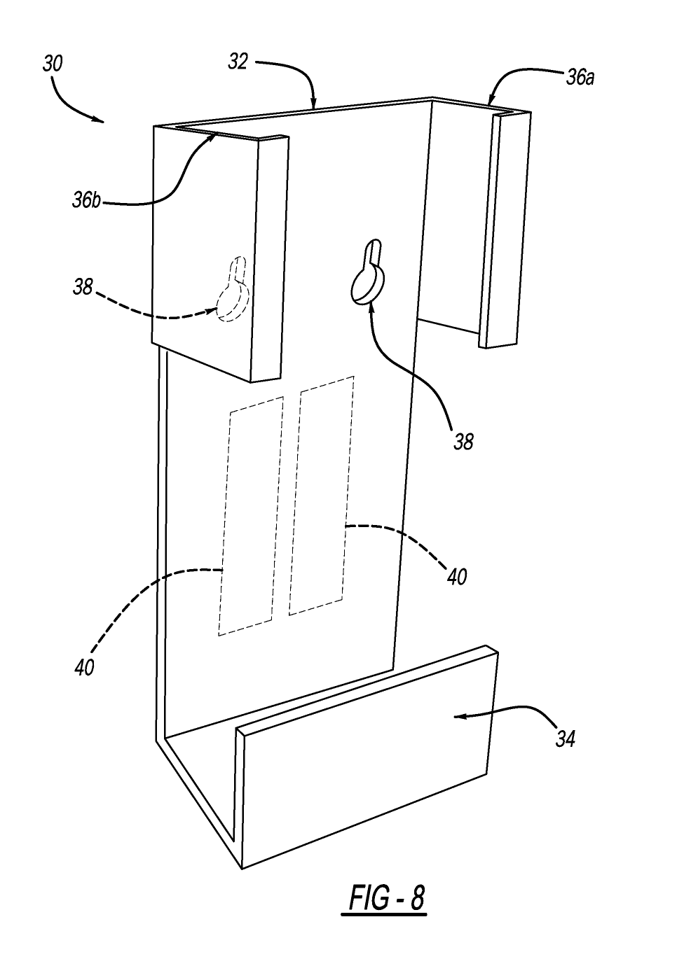

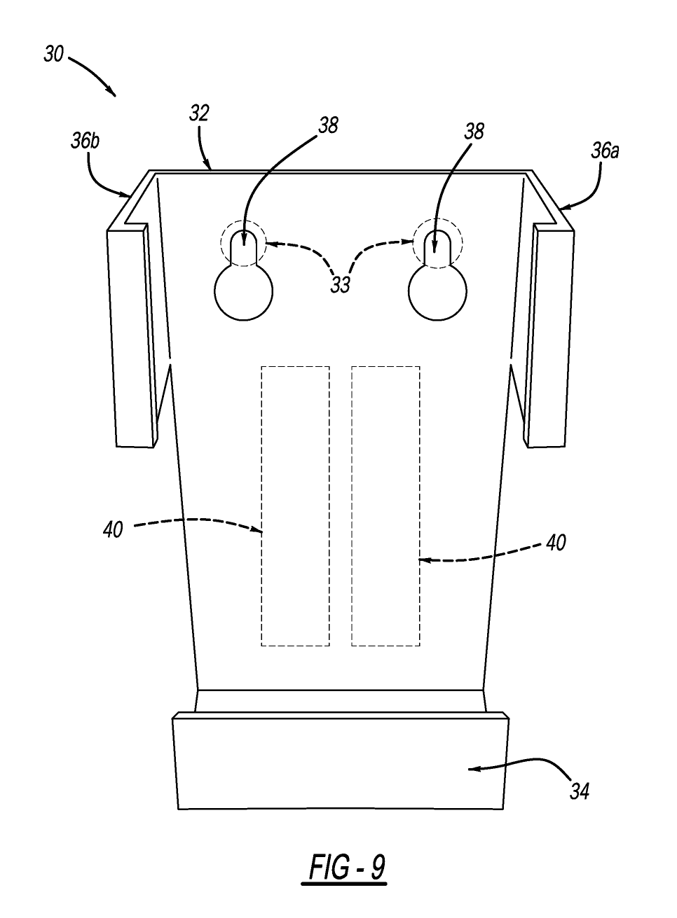

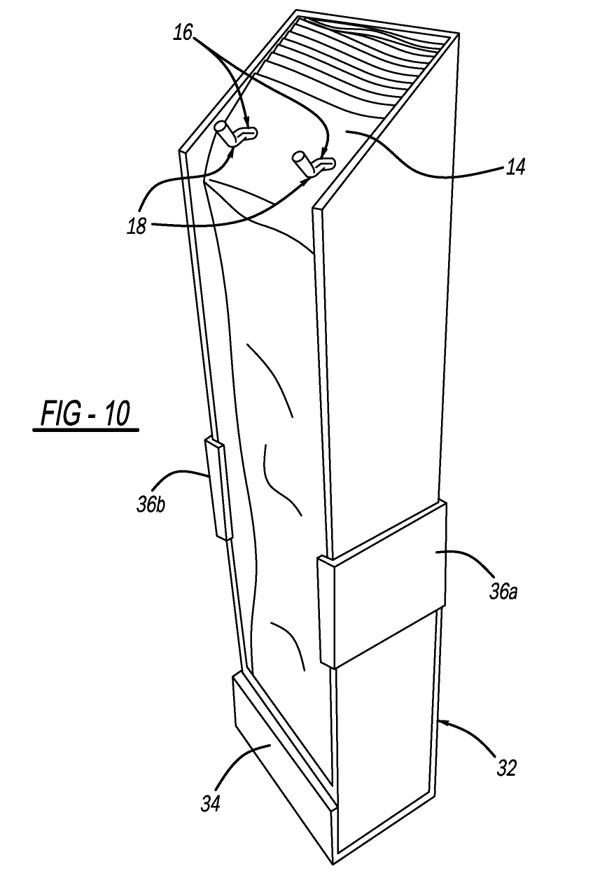

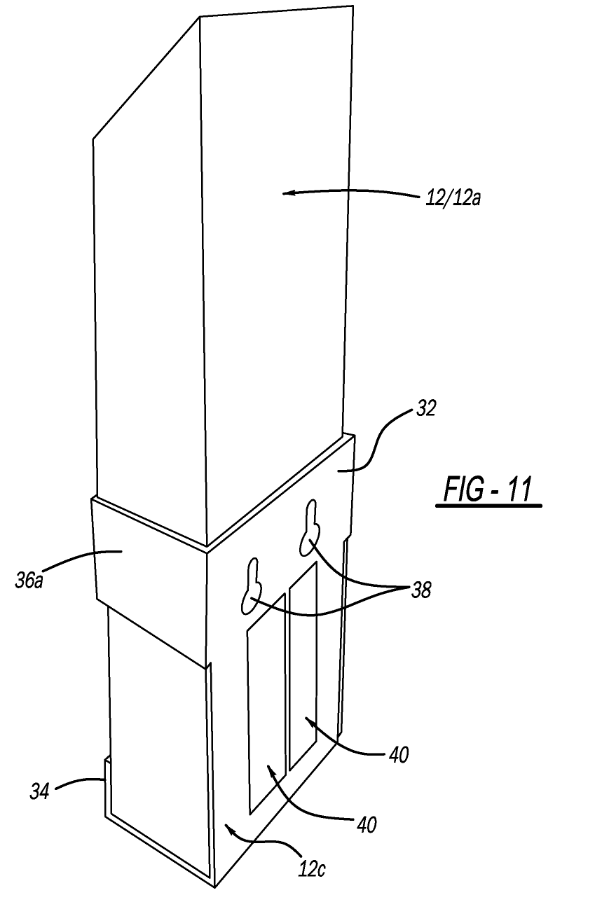

In yet another aspect of the invention, the housing 12a may be retained within a novel wall retainer 30. As shown in FIG. 8, the wall retainer 30 may contain: a rear wall 32 for retaining the retainer 30 on a desired structure; a lower or shorter front wall 34, opposite the rear wall 32, for retaining the housing 12 within the hanger 30; and a pair of opposed side walls 36a and 36b at least partially coextensive with and extending from an upper portion of the rear wall 32, also used to retain the housing 12 within the retainer 30. At least one orifice 38, and preferably a pair of orifices 38, may be formed within the rear wall 32, whereby a fastener 33 such as a nail or screw may extend there-through to secure the retainer 30 to a desired structure, such as a wall in a doctor's room, for example. Alternatively, at least one adhesive strip 40, or a pair of adhesive strips 40, may be affixed to the rear wall exterior 34b, and thereafter affixed to a desired structure such as a cabinet wall, to thereby suspend the retainer 30 for housing the polymeric sleeves 14. See FIGS. 8-11.

The present invention is exemplified and illustrated by the embodiments described herein. Those of ordinary skill in the art will realize that modifications to the novel aspects of the present invention are within the scope and spirit of the present invention, and inclusive herein.

What is claimed is:

1. A system for maintaining cleanliness of instruments comprising:
a housing containing an exterior and an interior, said exterior optionally configured to mount to a surface;
a rack contained within said housing; and
a plurality of flexible plastic covers, each cover configured to hang on said rack.

2. The system of claim 1 wherein said rack comprises at least one hangar.

3. The system of claim 2 wherein said at least one hangar comprises at least one dowel and each of said plurality of flexible plastic covers contains at least one aperture for receipt of said at least one dowel.

4. The system of claim 3 wherein said at least one dowel has an open upwardly-angled end for retaining said plurality of flexible plastic covers.

5. The system of claim 1 wherein said housing further contains an inner cavity defined by an outer wall, said rack and said plurality of flexible plastic covers contained therein.

6. The system of claim 5 wherein said housing contains a cover in slidable engagement with said outer wall, thereby enclosing said housing.

7. The system of claim 2 wherein each sleeve of said plurality of flexible plastic covers contains at least one aperture for receipt of said at least one hangar.

8. The system of claim 1 wherein each cover of said plurality of flexible plastic covers contains:
a first sleeve containing a first closed end and a first open end for receipt of an instrument; and
a second sleeve containing a second closed end attached to and adjacent to said first open end, and, a second open end opposite said second closed end for opening said first open end.

9. The system of claim 1 further comprising a retainer for containing said housing, said retainer configured to mount to a desired structure.

10. A system for maintaining cleanliness of instruments comprising:
a housing containing an exterior and an interior, said exterior configured to mount to a surface;
a rack containing at least one hangar, said rack contained within said housing; and
a plurality of flexible polymeric covers, each cover configured to hang on said at least one hangar,
wherein each cover of said plurality of flexible plastic covers contains a first sleeve containing a first closed end and a first open end for receipt of an instrument, and, a second sleeve containing a second closed end attached to and adjacent to said first open end, and, a second open end opposite said second closed end for opening said first open end.

11. The system of claim 10 wherein said rack comprises at least one hangar.

12. The system of claim 10 wherein said rack comprises at least one dowel and each of said plurality of flexible polymeric covers contains at least one aperture for receipt of said at least one dowel.

13. The system of claim 12 wherein said at least one dowel has an open upwardly-angled end.

14. The system of claim 9 wherein said rack comprises a pair of hangars.

15. The system of claim 13 wherein said pair of hangars comprises a pair of dowels or pins.

16. A system for maintaining cleanliness of instruments comprising:
a retainer configured for mounting to a desired structure;
a housing containing an exterior and an interior, said housing contained within said retainer;
a rack contained within said housing; and
a plurality of flexible polymeric covers, each cover configured to hang on said rack.

17. The system of claim 16 wherein each cover of said plurality of flexible polymeric covers contains:
a first sleeve containing a first closed end and a first open end for receipt of an instrument; and
a second sleeve containing a second closed end attached to and adjacent to said first open end, and, a second open end opposite said second closed end for opening said first open end.

18. The system of claim 16 wherein said rack comprises at least one dowel and each of said plurality of flexible polymeric covers contains at least one aperture for receipt of said at least one dowel.

19. The system of claim 18 wherein said at least one dowel has an open upwardly-angled end.

20. The system of claim 18 wherein said rack comprises a pair of dowels, and each of said plurality of flexible polymeric covers containing a corresponding pair of apertures, each of said dowels containing an open upwardly-angled end for receipt of one of said pair of apertures.
